(19) United States
(12) Patent Application Publication
(10) Pub. No.: US 2023/0118299 A1
(43) Pub. Date: Apr. 20, 2023

(54) RADIOLOGIST FINGERPRINTING

(71) Applicant: KONINKLIJKE PHILIPS N.V., EINDHOVEN (NL)

(72) Inventors: Tobias KLINDER, UELZEN (DE); Xin WANG, BELMONT, MA (US); Tanja NORDHOFF, HAMBURG (DE); Yuechen QIAN, LEXINGTON, MA (US); Vadiraj krishnamurthy HOMBAL, WAKEFIELD, MA (US); Eran RUBENS, PALO ALTO, CA (US); Sandeep Madhukar DALAL, WINCHESTER, MA (US); Axel SAALBACH, HAMBURG (DE); Rafael WIEMKER, KISDORF (DE)

(21) Appl. No.: 17/909,454

(22) PCT Filed: Mar. 4, 2021
(86) PCT No.: PCT/EP2021/055410

§ 371 Date: Sep. 6, 2022

Related U.S. Application Data
(60) Provisional application No. 62/986,881, filed on Mar. 9, 2020.

(51) Int. Cl. G16H 40/20; G16H 15/00; G06F 30/10

(52) U.S. Cl. CPC G16H 40/20; G16H 15/00; G06F 30/10

(57) ABSTRACT

An apparatus (10) for assessing radiologist performance includes at least one electronic processor (20) programmed to: during reading sessions in which a user is logged into a user interface (UI) (27), present (98) medical imaging examinations (31) via the UI, receive examination reports on the presented medical imaging examinations via the UI, and file the examination reports; and perform a tracking method (102, 202) including at least one of: (i) computing (204) concurrence scores (34) quantifying concurrence between clinical findings contained in the examination reports and corresponding computer-generated clinical findings for the presented medical imaging examinations which are generated by a computer aided diagnostic (CAD) process miming as a background process during the reading sessions; and/or (ii) determining (208) reading times (38) for the presented medical imaging examinations wherein the reading time for each presented medical imaging examination is the time interval between a start of the presenting of the medical imaging examination via the user interface and the filing of the corresponding examination report; and generating (104) at least one time-dependent user performance metric (36) for the user based on the computed concurrence scores and/or the determined reading times.

### FIELD

The following relates generally to the radiology arts, radiology examination reading arts, imaging workflow arts, computer-aided diagnostic (CAD) arts, and related arts.

### BACKGROUND

In the past few years, machine-learning (ML) or deep learning (DL) artificial intelligence (AI) solutions have reached or surpassed human-like performance levels for various tasks like detection of relevant findings (e.g., detection of lung nodules in computed tomography (CT) scans, breast lesions in mammograms, pneumothorax in chest X-ray, etc.). However, due to several reasons, most prominently regulatory issues, such solutions are not well integrated into clinical routines.

At the same time, radiologist performance assessment is increasingly requested in radiology as a way to ultimately improve throughput and accuracy of radiology examination readings, which can reduce costs, while maintaining or improving reading quality.

One performance metric is radiology report turnaround time (TAT), which is defined as the time interval between when the clinical images are uploaded to the radiology information system following the completion of the radiology examination by the technologist, and the time when the radiology examination report is finalized by the staff radiologist. TAT impacts the patient, the referring physicians and the entire hospital facility. Radiologists must be able to work around TAT for the best patient care purposes. It will be noted that the TAT depends on factors, which are at least partly outside of the radiologist's control, such as the backlog of radiology examinations to be read.

Of more relevance for assessing radiologist performance is the reading time, which is the time interval between when the radiologist opens a radiology examination to perform the reading and the time when the radiologist files the final radiology report containing the radiologist's findings. Reading time depends on both the radiologist and the procedure type. For example, reading time can be impacted by the complexity of the imaging examination (e.g., a complex three-dimensional CT for assessing cardiac health may take longer to read than a two-dimensional X-ray for assessing a possible bone fracture), the complexity of the patient context (e.g., if the patient has a complex medical history and/or a number of previous imaging examinations then the radiologist is expected to review this patient history so as to be informed of the patient context), and/or different working efficiencies of the individual radiologist at different time of a day and/or on different days of a week.

Currently, radiologists usually work in a Picture Archiving and Communication System (PACS)-driven workflow. A PACS workstation has a number of worklists, which are typically populated depending on examination status, location, modality and body part. A radiologist can select which case to read next from the worklist. With this “cherry-picking” case selection, some radiologists may tend to pick less complicated cases, which can lead to an accumulation of unread complicated cases at the end of the day or the shift. In addition, this ad-hoc based selection is not optimized for efficiency and quality. Moreover, urgency can be a factor in case selection, as critical scans should be read before non-critical scans.

Without an overall knowledge about how radiologist's reading efficiency varies across different procedure types through the day and week, any unusual reading performance cannot be determined, which results in not being able to dynamically manage it to avoid any possible back-log of studies and/or impacted reading quality. In addition, the radiologist's accuracy in correctly reading the selected cases is also an efficiency factor.

The following discloses certain improvements to overcome these problems and others.

### SUMMARY

In one aspect, an apparatus for assessing radiologist performance includes at least one electronic processor programmed to: during reading sessions in which a user is logged into a user interface (UI), present medical imaging examinations via the UI, receive examination reports on the presented medical imaging examinations via the UI, and file the examination reports; and perform a tracking method including at least one of: (i) computing concurrence scores quantifying concurrence between clinical findings contained in the examination reports and corresponding computer-generated clinical findings for the presented medical imaging examinations which are generated by a computer aided diagnostic (CAD) process running as a background process during the reading sessions; and/or (ii) determining reading times for the presented medical imaging examinations wherein the reading time for each presented medical imaging examination is the time interval between a start of the presenting of the medical imaging examination via the user interface and the filing of the corresponding examination report; and generating at least one time-dependent user performance metric for the user based on the computed concurrence scores and/or the determined reading times.

In another aspect, an apparatus for assessing radiologist performance includes at least one electronic processor programmed to: during reading sessions in which a user is logged into a UI, present medical imaging examinations via the UI including displaying medical images of the medical imaging examinations, and receive user-generated clinical findings via the UI for the presented medical imaging examinations; and perform a tracking method including: as a background process running during the reading sessions, performing a CAD process on the medical images of the presented medical imaging examinations to generate computer-generated clinical findings for the presented medical imaging examinations; and computing concurrence scores quantifying concurrence between the computer-generated clinical findings for the presented medical imaging examinations and the corresponding user-generated clinical findings for the presented medical imaging examinations; and generating a time-dependent user performance metric for the user based on the concurrence scores.

In another aspect, an apparatus for assessing radiologist performance includes at least one electronic processor programmed to perform a method during reading sessions in which a user is logged into a UI includes: providing a worklist of unread medical imaging examinations via the UI, presenting medical imaging examinations selected from the worklist by the user via the UI, receiving examination reports via the UI for the presented medical imaging examinations, and filing the received examination reports; determining a reading time for each presented medical imaging examination as the time interval between a start of the presenting of the medical imaging examination via the UI and the filing of the corresponding received examination report; and generating a time-dependent user performance metric for the user based on the determined reading times.

One advantage resides in providing a comparison between a performance of an individual radiologist performing one or more imaging studies against AI-enabled algorithms performing the same or similar imaging studies.

Another advantage resides in running background programs to track similarities between the radiologist's performance and the AI-enabled algorithms.

Another advantage resides in not using the results of AI-enabled algorithms in patient diagnoses.

Another advantage resides in tracking a performance of a radiologist during imaging studies to obtain a benchmark level of performance of the radiologist.

Another advantage resides in tracking an accuracy performance of a radiologist during imaging studies to obtain a benchmark accuracy level of performance of the radiologist.

Another advantage resides in obtaining the benchmark level of performance of the radiologist as an internal reference.

Another advantage resides in determining an efficiency of a radiologist performing medical imaging examinations based on reading times of the radiologist.

Another advantage resides in updating a schedule or workflow of the radiologist based on reading times of the radiologist.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative apparatus for assessing radiologist performance in accordance with the present disclosure.

FIG. 2 shows exemplary flow chart operations performed by the apparatus of FIG. 1.

### DETAILED DESCRIPTION

As used herein, the term “background process” (and variants thereof) refers to a computer process that runs autonomously without user intervention behind the scenes of another process (such as an imaging reading session).

As used herein, the term “concurrence score” (and variants thereof) refers to a relationship between results of an imaging reading session by a radiologist and results generated by an Al background process.

As used herein, the term “fingerprint” (and variants thereof) refers to a relationship between personal reading characteristics of a radiologist and potentially small differences relative to other radiologists.

As used herein, the term “user performance metric” (and variants thereof) refers to a timestamping or fitting process of the fingerprint or concurrence score.

AI-based systems, such as Computer Aided Diagnostic (CAD) systems, are becoming highly accurate, and in principle are usable for clinical diagnostic tasks. Such use is however inhibited by non-technical considerations, such as that regulatory frameworks may not permit CAD for diagnosis, or if permitted, incorporating CAD would require costly recertification of systems and processes for regulatory approval.

The following discloses, in some embodiments, running AI CAD programs in the background. The AI CAD results are not used to provide or aid in actual diagnoses. Rather, the AI CAD results are compared with the clinical findings contained in the radiology examination report prepared by the radiologist, in order to generate a concurrence score, sometimes referred to in these embodiments as a fingerprint, for the radiologist, which measures how well the radiologist's clinical findings concur with the AI CAD generated clinical findings. Assuming the AI CAD is reasonably accurate, it can be expected that higher concurrence scoring correlates with higher accuracy in radiology readings by the radiologist. This will remain true so long as the AI CAD is reasonably accurate. Hence, there is no requirement that the AI CAD be perfect or of sufficient accuracy for clinical diagnosis. The concurrence score for a radiologist may be computed as a function of time, and may be broken up in various ways, e.g. different concurrence scores for different types of readings.

There can be various uses for the concurrence score. It may be used to track the radiologist's performance over the day to identify time periods when the radiologist's accuracy may lag (e.g. late afternoon due to fatigue). It can be used to compare performance of radiologists across a department or between hospitals. Shifts in the concurrence score may also be an indicator of an issue in the radiology reading process. For example, reduced concurrence scores across all radiologists could be due to changes in the imaging protocol or an equipment malfunction (which could lead to the AI CAD accuracy decreasing). Advantageously, these embodiments leverage the AI CAD in actual clinical workflow, while avoiding the regulatory or other non-technical considerations that have conventionally limited or prevented use of AI CAD in clinical diagnosis of actual patients.

In other (not necessarily mutually exclusive) embodiments disclosed herein, a different type of radiologist fingerprint is provided to assess efficiency of radiology readings. In these embodiments, the fingerprint is a metric of how often the radiologist fails to meet expected reading times for examinations. This assessment leverages the fact that most PACS implementations timestamp the beginning of a radiology examination reading (when the radiologist accesses the imaging examination data) and the end of the reading (when the radiology report is filed), with the reading time being in between. To establish “expected” reading times (e.g., on an individual radiologist basis), the reading times of each radiologist are analyzed statistically to determine a typical reading time threshold that the radiologist usually meets. For higher granularity, the reading time thresholds are preferably determined for specific reading tasks (e.g. the reading time threshold for a simple CT reading to detect a possible bone fracture may be much shorter than the reading time threshold for a complex PET scan reading to detect possible lesions), and may also be determined for specific days of the week, specific parts of the day, or other specific time periods (e.g., the radiologist may be less efficient on Mondays compared with Tuesdays; or may be more efficient in afternoons compared with mornings or vice versa).

After this setup, the radiologist's reading time for each reading is compared with the reading time threshold for that radiologist and that type of reading (and optionally for that day of week, etc.). If more than a certain number of readings per time block are over threshold (e.g., more than 2 readings in a 30 minute period are over reading time threshold in one example), then the over-threshold readings are assessed as to patient context. If there is something in the patient context that justifies the longer reading times, then this over-threshold reading time is discounted. If, after this patient context analysis, the number of over-threshold reading times in the time block is still too high, then a dynamic management of the radiologist's workload is invoked.

The dynamic management may, for example, include assigning the radiologist some easier readings. Alternatively, if the radiologist is performing well (no over-threshold reading times over the most recent time block(s)), then that radiologist may be assigned some more challenging readings since the reader is shown as being preferred reader for these types of images. More generally, the over-threshold fingerprints of the radiologists can be used to intelligently distribute unread cases to the available radiologists.

In existing radiology reading systems, the radiologist is usually presented with a queue of pending cases. This can lead to cherry-picking of the easier cases. The dynamic management can additionally or alternatively be implemented by adjusting the pending cases queue on an individual radiologist basis so that the radiologist is presented with only the appropriate cases based on the radiologists' current reading time performances on readings of different types.

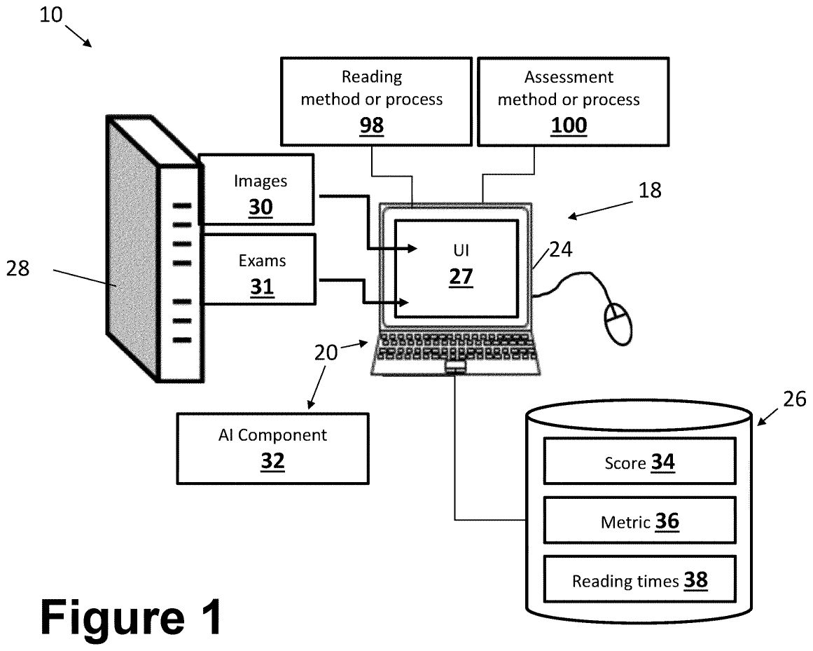

With reference to FIG. 1, an illustrative apparatus 10 is shown for assessing radiologist performance for reviewing images generated by an image acquisition device (not shown). FIG. 1 also shows an electronic processing device 18, such as a workstation computer, or more generally a computer. The electronic processing device 18 typically includes a radiology reading workstation, and may also include a server computer or a plurality of server computers, e.g. interconnected to form a server cluster, cloud computing resource, or so forth, to perform more complex image processing or other complex computational tasks. The workstation 18 includes typical components, such as an electronic processor 20 (e.g., a microprocessor), at least one user input device (e.g., a mouse, a keyboard, a trackball, and/or the like) 22, and a display device 24 (e.g. an LCD display, plasma display, cathode ray tube display, and/or so forth). In some embodiments, the display device 24 can be a separate component from the workstation 18, or may include two or more display devices (e.g., a high resolution display for presenting clinical images of the radiology examination, and a lower resolution display for providing textual or lower-resolution graphical content).

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 27 for display on the display device 24.

The apparatus 10 also includes, or is otherwise in operable communication with, a database 28 storing a set 30 of images and/or medical imaging examinations 31 to be reviewed. The database 28 can be any suitable database, including a Radiology Information System (RIS) database, a Picture Archiving and Communication System (PACS) database, an Electronic Medical Records (EMR) database, and so forth. In particular, the database 28 typical comprises a PACS database or functional equivalent thereof. Alternatively, the database 28 can be implemented in the non-transitory medium or media 26. The workstation 18 can be used to access the stored set 30 of images of the radiology examination 31 to be read, along with imaging metadata, for example stored in DICOM format.

The images 30 can be downloaded to the workstation 18 from the database 28 so that the radiologist can review the images and report findings (e.g., presence of a lesion, errors in the image, regions of interest in the images, and so forth). In some embodiments, the at least one electronic processor 20 is further programmed to implement an AI component 32. The AI component 32 is programmed to run one or more algorithms (e.g., CAD algorithms) on the set 30 of images as the radiologist reviews the image so as to generate computer-generated clinical findings for the presented medical imaging examinations 31. However, unlike in a typical CAD system, the computer-generated clinical findings are not presented to the radiologist for consideration in performing the reading of the radiology examination 31. Rather, the at least one electronic processor 20 is programmed to compute a fingerprint or concurrence score 34 based on a comparison between the performance of the radiologist and the AI component 32. From the concurrence scores 34, a user performance metric 36 is computed for the radiologist. In this way, the AI component 32 does not play any role in the clinical radiology reading process (e.g., the computer-generated clinical findings are not known to the radiologist performing the reading, and are not included in the filed radiology report). As a consequence, the AI component 32, and its use as disclosed herein, typically does not require regulatory approval by medical regulatory authority.

In other (not necessarily mutually exclusive) embodiments, a radiologist fingerprint is generated based on the tracking of reading times, and may be used for example in dynamic management of the radiologist's workload, as further described herein.

The apparatus 10 is configured as described above to perform a radiology reading method 98 and a radiologist performance assessment method or process 100. The non-transitory storage medium 26 stores instructions which are readable and executable by the at least one electronic processor 20 to perform disclosed operations including performing the reading method 98 and the radiologist performance assessment method or process 100. In some examples, one or both of the methods 98, 100 may be performed at least in part by cloud processing.

The radiology reading method 98 provides the radiologist with the tools for reading radiology examinations. In a typical workflow, the radiologist logs into the workstation 18 in order to conduct a reading session. The login may be done by the radiologist entering his or her username and password. In other login approaches, a biometric-based login may be employed, e.g. using a fingerprint reader (not shown) that reads a fingerprint on a finger of the radiologist, or using facial recognition, or so forth. Other typical login approaches can be utilized, e.g. two-factor authorization in which the radiologist enters a password and also inserts a USB security key, provides a computer-generated one-time passcode, or so forth.

During the reading sessions, the user (e.g., radiologist) is logged into the UI 27. The user selects a medical imaging examination 31 from the worklist provided by the UI 27, and the selected medical imaging examinations is presented via the UI 27. This presentation may, for example, include operations such as displaying clinical images 30 of the examination on the display device 24 and enabling the user to zoom, pan, or otherwise manipulate the display of the images. The UI 27 may provide other functionality such as allowing the user to manipulate on-screen cursors for measuring distances in the images, delineating lesions or other features of interest, and so forth. The UI 27 also provides a user input window via which an examination report is received on the presented medical imaging examinations 31 via the UI 27. The user (e.g. radiologist) writes up the radiology report including providing the radiologist's clinical findings. When the report is complete, the user files the examination report, e.g. by uploading the final report to the PACS database 28. The radiology reading method 98 may, for example, be implemented as a commercially available radiology reading environment such as the IntelliSpace PACS Radiology reading environment (available from Koninklijke Philips N.V., Eindhoven, the Netherlands).

In a typical radiology department, the radiologist logs into a workstation 18 at the start of each day's work shift, and conducts a reading session, which may include performing readings of a number of radiology examinations. The radiologist logs out at the end of the work shift (and may also log out/back in at other intervals, such as in order to take a lunchbreak). The radiologist thereby conducts successive reading sessions, which may extend over days, weeks, months, or years depending upon the radiologist's tenure at the radiology department. The performance of the radiologist in these successive reading sessions is assessed by a radiologist performance assessment method 100, embodiments of which are described herein.

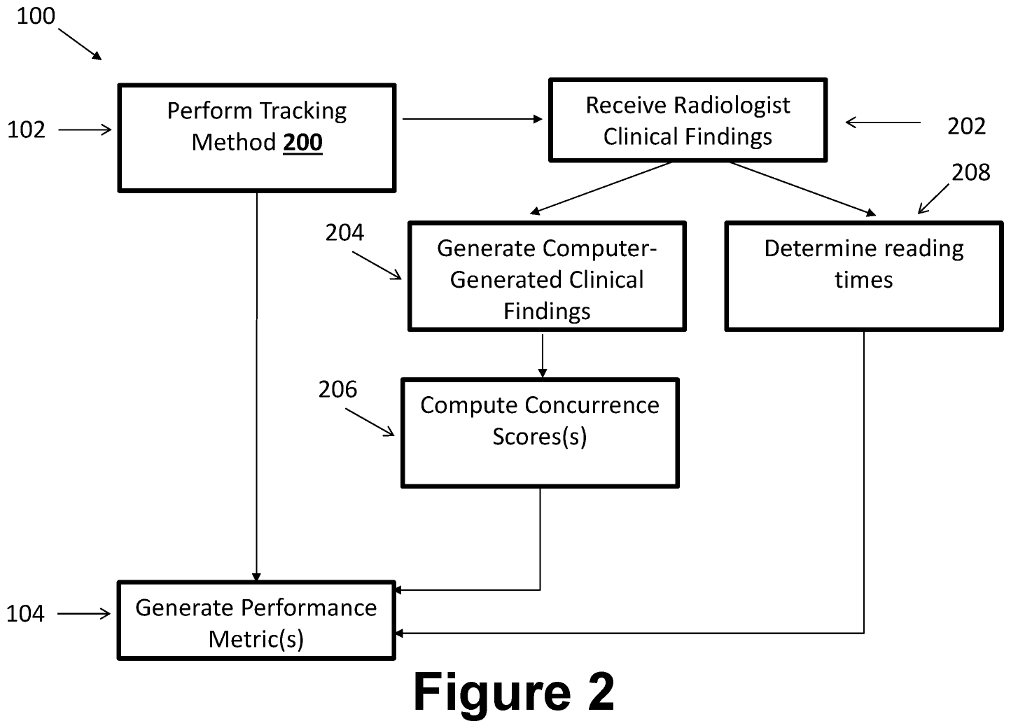

With continuing reference to FIG. 1 and with further reference to FIG. 2, an illustrative embodiment of the radiologist performance assessment method 100 is diagrammatically shown as a flowchart 100 in FIG. 2. At an operation 102, the at least one electronic processor 20 is programmed to perform a tracking method 200 during successive reading sessions which the user is logged in to the GUI 27 and conducting radiology examination readings per the reading method 98.

In one embodiment, the tracking method 200 can include operations 202-206. At an operation 202 (which is actually performed by the reading method 98), the medical imaging examinations 31 are presented on the GUI 27, including displaying the medical images of the imaging sessions. The user then inputs, via the at least one user input device 22, clinical findings (e.g., presence of a lesion, errors in the image, regions of interest in the images, and so forth) via the GUI 27 for the medical imaging examinations 31.

At an operation 204, which is run concurrently in the background with the operation 202, the at least one electronic processor 20 is programmed to perform a CAD process on the medical images of the presented medical imaging examinations 31. In some embodiments, the AI component 32 performs the operation 204 as an AI-CAD process. The CAD process generates computer-generated clinical findings for the medical examinations presented to the user at the operation 202. Advantageously, the computer-generated clinical findings are not presented to the user when the user is logged in to the GUI 27. Thus, the computer-generated clinical findings are not used in diagnoses.

At an operation 206, the at least one electronic processor 20 is programmed to extract clinical findings entered by the user per operation 202, and compute the one or more concurrence scores 34. The concurrence scores 34 quantify a concurrence (e.g., similarity) between the computer-generated clinical findings for the presented medical imaging examinations 31 and the corresponding user-generated clinical findings for the presented medical imaging examinations.

The user-generated clinical findings can be identified in various ways. In one approach, the radiology report entered by the user in the operation 202 is processed to extract the user-generated clinical findings. The method for extracting the user-generated clinical findings from the report depends upon the format of the report. If the findings are input to the report in a structured data field or fields of the report designated for entry of findings, then the user-generated clinical findings may be extracted simply by reading the clinical findings from the data field(s) designated for entry of clinical findings. On the other hand, if the findings are input into the report in freeform entry fields, then the extraction may entail natural language processing (NLP) techniques such as detecting keywords associated with clinical findings and/or performing semantic analysis of the text. For example, in the freeform text entry “Lesion size increased to 1.25 mm” the terms “lesion”, “size”, and “increased” may be detected to extract the finding “lesion size increasing”, while the additional content “1.25 mm” may allow extraction of the finding “lesion size=1.25 mm”. These are merely non-limiting illustrative examples. Once the concurrence scores 34 are calculated, the tracking method 200 is complete.

As an operation 104, the at least one electronic processor 20 is programmed to generate one or more user performance metrics 36 for the user based on the concurrence scores 34 computed over the successive reading sessions. In some embodiments, the user performance metric 36 is time-dependent. For example, the user performance metric 36 can be a time sequence of timestamped concurrence scores 34. In another example, the user performance metric 36 can include a post-processing operation, such as fitting the concurrence scores 34 as a function of time to a graphical representation, such as a polynomial function. In other embodiments, a plurality of finding-type, time-dependent user performance metrics 36 can be generated by performing the tracking method 200 using different finding-type specific CAD processes running as background processes. In further embodiments, the at least one electronic processor 20 is programmed to analyze the time-dependent user performance metric 36 on a per-day time interval to identify one or more time intervals in which the time-dependent user performance metric falls below a threshold. If the user performance metric falls below the threshold, certain remedial actions can be taken (e.g., adjusting a schedule of the radiologist, reviewing the tracking method 200 to see if a process error exists, and so forth).

In some embodiments, the tracking method 200 is repeated for multiple, different radiologists, for which individual user-specific time-dependent user performance metrics 36 can be generated. The at least one electronic processor 20 is programmed to compare performance of the different users by displaying, on the display device 24, a comparison (e.g., numerical, graphical, and so forth), of the different user-specific time-dependent user performance metrics 36.

With continuing reference to FIGS. 1 and 2, in another embodiment, instead of, or in addition to, running background CAD processes and performing the operations 204, 206, the tracking method 200 can include determining reading times 38 of the medical imaging examinations 31 by the radiologist. A fingerprint or user performance metric 36 can be generated for the radiologist(s) based on reading times of past readings, reading time based on procedure type, how reading time varies at different rimes of a workday or different days of a week, a patient context for each patient in the medical imaging examination, and so forth. As used herein, the term “patient context” (and variants thereof) refers to a complexity of various factors, such as different reasons for previous visits for the patient, the number of previous visits, and the number of scans taken in the past for the same procedure type, etc.

To determine the reading times 38, the tracking method 200 includes the operation 208. At the operation 202, as already described the medical examinations are retrieved from the database 28 and presented via the GUI 27 as a worklist of unread examinations. The user can select the examinations for review. The reviewed examination reports can be filed (e.g., stored) in the database 28. (Again, the operation 202 corresponding to the reading method or process 98 indicated in FIG. 1).

At the operation 208, the at least one electronic processor 20 is programmed to determine a reading time 38 for each presented medical imaging examination 31 as the time interval between a start of the presenting of the medical imaging examination via the GUI 27 and the filing of the corresponding received examination report. The reading times 38 can be stored in the non-transitory computer readable medium 26 and/or displayed on the display device 24.

In this embodiment, the operation 104 includes generating the time-dependent user performance metric 36 for the user based on the reading times 38 over successive reading sessions. In some embodiments, the user performance metric 36 is time-dependent. For example, the user performance metric 36 can be a time sequence of timestamped concurrence scores 34. In another example, the user performance metric 36 can include a post-processing operation, such as fitting the concurrence scores 34 as a function of time to a graphical representation, such as a polynomial function. In other embodiments, a plurality of finding-type, time-dependent user performance metrics 36 can be generated by performing the tracking method 200 using reading times 38 for different types of medical imaging examinations 31. In some embodiments, the tracking method 200 is repeated for multiple, different radiologists, for which individual user-specific time-dependent user performance metrics 36 can be generated. The at least one electronic processor 20 is programmed to compare performance of the different users by displaying, on the display device 24, a comparison (e.g., numerical, graphical, and so forth), of the different user-specific time-dependent user performance metrics 36.

In further embodiments, the at least one electronic processor 20 is programmed to analyze the time-dependent user performance metric 36 on a per-day time interval to identify one or more time intervals in which the time-dependent user performance metric falls below or underruns a threshold based at least on a patient context of the images reviewed to generate the time-dependent user performance metric. For example, if a radiologist's reading time exceeds the pre-defined threshold, the at least one electronic processor 20 is programmed to assess the patient context, and automatically flag and trigger a check on the patient's context. If the patient's context is significantly complex, the at least one electronic processor 20 is programmed to determine that his long reading time is due to the complex patient context; otherwise, the at least one electronic processor determines that current reading performance of the radiologist is unusual.

If the user performance metric falls below the threshold, certain remedial actions can be taken (e.g., adjusting a schedule of the radiologist, reviewing the tracking method 200 to see if a process error exists, and so forth). For example, after a pre-defined number of unusual behavior cases were detected within a certain amount of time (e.g., 2 cases within 30 minutes), the at least one electronic processor 20 is programmed to dynamically adjust a reading schedule of the radiologist, such as assigning the radiologist a fewer number of cases than usual, or assigning less complicated cases (such as chest x-ray), and adjusting other radiologists' reading assignments accordingly as needed in order to not slowing down the overall throughput.

In a particular example, for an imaging examination comprising a CT scan of a patient's head without contrast, the maximum reading time of a particular radiologist during 8-10 AM on Monday is 9 minutes. If this maximum reading time is set as the detection threshold for this particular radiologist, and one Monday morning, the reading time at 9 AM is 11 minutes, this performance is flagged as unusual after a confirmation that the patient's context is not significantly complex. After the pre-defined number of unusual behavior cases were detected within the pre-defined amount of time, the schedule of the particular radiologist can be adjusted accordingly (e.g., to include fewer cases or less complex cases). In addition, the schedules of the other radiologists can also be updated to account for the changes in the particular radiologist's schedule.

In some examples, the AI component 32 can be configured with a self-learning component, in that the AI component is configured to assess the user performance metric 36 for one or more radiologists based on imaging protocols, reading preferences and so forth. For example, for a spectral CT imaging protocol, the AI component 32 is configured to update the user performance metric 36 based on the results of the radiologist (e.g., the radiologist's performance is more consistent with the AI-CAD process when MonoE images are reviewed as opposed to conventional CT images).

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An apparatus for assessing radiologist performance, the apparatus comprising at least one electronic processor programmed to:
during reading sessions in which a user is logged into a user interface (UI), present medical imaging examinations via the UI, receive examination reports on the presented medical imaging examinations via the UI, and file the examination reports; and
perform a tracking method including at least one of:
(i) computing concurrence scores quantifying concurrence between clinical findings contained in the examination reports and corresponding computer-generated clinical findings for the presented medical imaging examinations which are generated by a computer aided diagnostic (CAD) process running as a background process during the reading sessions; and/or
(ii) determining reading times for the presented medical imaging examinations wherein the reading time for each presented medical imaging examination is the time interval between a start of the presenting of the medical imaging examination via the user interface and the filing of the corresponding examination report; and
generating at least one time-dependent user performance metric for the user based on the computed concurrence scores and/or the determined reading times.

2. The apparatus of claim 1, wherein the tracking method further includes:
computing concurrence scores quantifying concurrence between the clinical findings contained in the examination reports and corresponding computer-generated clinical findings for the presented medical imaging examinations which are generated by a CAD process running as a background process during the reading sessions, and the generating includes generating a time-dependent user performance metric for the user based on the computed concurrence scores.

3. The apparatus of claim 2, wherein the generating includes:
generating a plurality of finding-type specific time-dependent user performance metrics by performing the tracking method using different finding-type specific CAD processes running as background processes.

4. The apparatus of claim 2, wherein the at least one electronic processor is not programmed to:
present the computer-generated clinical findings via the UI during the reading sessions in which the user is logged into the UI.

5. The apparatus of claim 1, wherein the at least one electronic processor is further programmed to:
analyze the time-dependent user performance metric on a per-day time interval to identify one or more time intervals in which the time-dependent user performance metric falls below a threshold.

6. The apparatus of claim 1, wherein the at least one electronic processor is programmed to repeat the performing of the tracking method for different users and to generate user-specific time-dependent user performance metrics for the different users, and is further programmed to:
compare performance of the different users by displaying a comparison of the user-specific time-dependent user performance metrics.

7. The apparatus of claim 1, wherein the CAD comprises an artificial intelligence (AI)-CAD.

8. The apparatus of claim 1, wherein the tracking method includes determining reading times for the presented medical imaging examinations wherein the reading time for each presented medical imaging examination is the time interval between a start of the presenting of the medical imaging examination via the user interface and the filing of the corresponding examination report, and the generating includes generating a time-dependent user performance metric for the user based on the determined reading times.

9. The apparatus of claim 8, wherein the at least one electronic processor is programmed to:
generate a plurality of finding-type specific time-dependent user performance metrics by performing the tracking method using different examination-types of medical imaging examinations.

10. The apparatus of claim 8, wherein the at least one electronic processor is further programmed to:
analyze the time-dependent user performance metric on per-day time interval to identify one or more time intervals in which the time-dependent user performance metric falls below a threshold.

11. The apparatus of claim 8, wherein the at least one electronic processor is programmed to repeat the performing of the tracking method for different users and to generate user-specific time-dependent user performance metrics for the different users, and is further programmed to:
compare performance of the different users by displaying a comparison of the user-specific time-dependent user performance metrics.

12. The apparatus of claim 8, wherein the at least one electronic processor is programmed to:
analyze the time-dependent user performance metric to determine when the time-dependent user performance metric underrun a predetermined quality threshold based on an assessment of a patient context of the images reviewed to generate the time-dependent user performance metric; and
altering a work schedule of the radiologist if the time-dependent user performance metric underruns the predetermined quality threshold after discounting patient context factors during the reading of the images of the medical imaging examinations.

13. The apparatus of claim 12, wherein the altering includes one or more of:
adding or removing cases from the work schedule of the radiologist;
generating the work schedule for the radiologist based on the at least one time-dependent user performance metric of the radiologist.

14. An apparatus for assessing radiologist performance, the apparatus comprising at least one electronic processor programmed to:
during reading sessions in which a user is logged into a user interface (UI), present medical imaging examinations via the UI including displaying medical images of the medical imaging examinations, and receive user-generated clinical findings via the UI for the presented medical imaging examinations; and
perform a tracking method including:
as a background process running during the reading sessions, performing a computer aided diagnostic (CAD) process on the medical images of the presented medical imaging examinations to generate computer-generated clinical findings for the presented medical imaging examinations; and
computing concurrence scores quantifying concurrence between the computer-generated clinical findings for the presented medical imaging examinations and the corresponding user-generated clinical findings for the presented medical imaging examinations; and
generating a time-dependent user performance metric for the user based on the concurrence scores.

15. The apparatus of claim 14, wherein the at least one electronic processor is programmed to:
generate a plurality of finding-type specific time-dependent user performance metrics by performing the tracking method using different finding-type specific CAD processes running as background processes.

16. The apparatus of claim 14, wherein the at least one electronic processor is further programmed to:
analyze the time-dependent user performance metric on a per-day time interval to identify one or more time intervals in which the time-dependent user performance metric falls below a threshold.

17. The apparatus of claim 14, wherein the at least one electronic processor is programmed to repeat the performing of the tracking method for different users and to generate user-specific time-dependent user performance metrics for the different users, and is further programmed to:
compare performance of the different users by displaying a comparison of the user-specific time-dependent user performance metrics.

18. An apparatus for assessing radiologist performance, the apparatus comprising at least one electronic processor programmed to perform a method during reading sessions in which a user is logged into a user interface (UI), the method including:
providing a worklist of unread medical imaging examinations via the UI, presenting medical imaging examinations selected from the worklist by the user via the UI, receiving examination reports via the UI for the presented medical imaging examinations, and filing the received examination reports;
determining a reading time for each presented medical imaging examination as the time interval between a start of the presenting of the medical imaging examination via the UI and the filing of the corresponding received examination report; and
generating a time-dependent user performance metric for the user based on the determined reading times.

19. The apparatus of claim 18, wherein the at least one electronic processor is programmed to:
analyze the time-dependent user performance metric to determine when the time-dependent user performance metric underrun a predetermined quality threshold based on a patient context of the images reviewed to generate the time-dependent user performance metric; and
altering a work schedule of the radiologist if the time-dependent user performance metric underruns the predetermined quality threshold after discounting patient context factors during the reading of the images of the medical imaging examinations.

20. The apparatus of claim 19, wherein the altering includes one or more of:
adding or removing cases from the work schedule of the radiologist;
generating the work schedule for the radiologist based on the time-dependent user performance metric of the radiologist.
